(19) United States
(12) Patent Application Publication
(10) Pub. No.: US 2022/0196646 A1
(43) Pub. Date: Jun. 23, 2022

(54) METHOD FOR MANUFACTURING PROTEIN BIOELECTRONIC DEVICES

(71) Applicant: Arizona Board of Regents on behalf of Arizona State University, Scottsdale, AZ (US)

(72) Inventors: Stuart Lindsay, Scottsdale, AZ (US); Eathen Ryan, Scottsdale, AZ (US)

(21) Appl. No.: 17/553,392

(22) Filed: Dec. 16, 2021

Related U.S. Application Data
(60) Provisional application No. 63/127,425, filed on Dec. 18, 2020.

(51) Int. Cl. G01N 33/543; G01N 27/04

(52) U.S. Cl. CPC G01N 33/5438; G01N 27/04

(57) ABSTRACT

The present disclosure provides devices, systems, and methods related to protein bioelectronics. In particular, the present disclosure provides devices, systems, and methods for forming electrical contacts to a protein with high yield, which facilitates the manufacture of analytical devices to detect and measure the electrical characteristics corresponding to protein function.

### RELATED APPLICATIONS

This application claims priority to and the benefit of U.S. Provisional Patent Application No. 63/127,425 filed Dec. 18, 2020, which is incorporated herein by reference in its entirety for all purposes.

### GOVERNMENT SUPPORT

This invention was made with government support under R01 HG011079 awarded by the National Institutes of Health. The government has certain rights in the invention.

### FIELD

The present disclosure provides devices, systems, and methods related to protein bioelectronics. In particular, the present disclosure provides devices, systems, and methods for forming electrical contacts to a protein with high yield, which facilitates the manufacture of analytical devices to detect and measure the electrical characteristics corresponding to protein function.

### BACKGROUND

As proteins perform their various functions, movements are generated that underlie these functions. The ability to develop devices, systems, and methods that measure the electrical characteristics corresponding to the fluctuations generated by an active protein can be a basis for label-free detection and analysis of protein function. For example, monitoring the functional fluctuations of an active enzyme may provide a rapid and simple method of screening candidate drug molecules that affect the enzyme's function. In other cases, the ability to monitor the fluctuations of proteins that process biopolymers (e.g., carbohydrates, polypeptides, nucleic acids, and the like) may reveal new information about their conformational changes and how those changes are linked to function. Additionally, diagnostic and analytical devices can be developed to take advantage of the electrical characteristics produced by active proteins, providing new ways to leverage biomechanical properties for practical use.

### SUMMARY

Embodiments of the present disclosure include a method of manufacturing a device for direct measurement of protein activity. In accordance with these embodiments, the method includes combining a first and second electrode with a protein-of-interest to form an electrical connection between the electrodes, wherein the first and second electrodes comprise surfaces chemically modified with a linker molecule, and wherein the protein-of-interest comprises at least one non-canonical amino acid. In some embodiments, applying a voltage bias to the electrodes produces current flow through the protein-of-interest.

In some embodiments, fluctuations in activity of the protein-of-interest correspond to fluctuations in current.

In some embodiments, the surfaces of the first and second electrodes are chemically modified with at least one thiolated biotin linker molecule.

In some embodiments, the at least one non-canonical amino acid comprises biotin or a derivative thereof. In some embodiments, the at least one non-canonical amino acid is biocytin or a derivative thereof. In some embodiments, the protein-of-interest comprises two biocytin non-canonical amino acids or derivatives thereof.

In some embodiments, the protein-of-interest comprises an Avitag sequence or a derivative thereof. In some embodiments, the protein-of-interest does not comprise an Avitag sequence or a derivative thereof.

In some embodiments, the method further comprises adding a second linker molecule to form the electrical connection. In some embodiments, the second linker molecule comprises a streptavidin molecule. In some embodiments, the streptavidin molecule comprises at least two biotin binding sites.

In some embodiments, the protein-of-interest comprises the least one non-canonical amino acid at two distinct locations. In some embodiments, the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

In some embodiments, the protein-of-interest is selected from the group consisting of a polymerase, a nuclease, a proteasome, a glycopeptidase, a glycosidase, a kinase and an endonuclease. In some embodiments, the protein-of-interest is a polymerase. In some embodiments, the exonuclease activity of the polymerase is disabled.

Embodiments of the present disclosure also include a device for direct measurement of protein activity. In accordance with these embodiments, the device includes a first electrode and a second electrode, wherein the first and second electrodes comprise surfaces chemically modified with at least one thiolated biotin linker molecule, and a protein-of-interest that forms an electrical connection between the first and second electrodes comprising at least one non-canonical amino acid, wherein the at least one non-canonical amino acid comprises biotin or a derivative thereof. In some embodiments, applying a voltage bias to the electrodes produces current flow through the protein-of-interest.

In some embodiments, fluctuations in activity of the protein-of-interest correspond to fluctuations in current.

In some embodiments, the at least one non-canonical amino acid is biocytin or a derivative thereof.

In some embodiments, the protein-of-interest comprises two biocytin non-canonical amino acids or derivatives thereof.

In some embodiments, the protein-of-interest comprises an Avitag sequence or a derivative thereof. In some embodiments, the protein-of-interest does not comprise an Avitag sequence or a derivative thereof.

In some embodiments, the device further comprises a second linker molecule comprising a streptavidin molecule.

In some embodiments, the protein-of-interest comprises the least one non-canonical amino acid at two distinct locations. In some embodiments, the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

In some embodiments, the protein-of-interest is selected from the group consisting of a polymerase, a nuclease, a proteasome, a glycopeptidase, a glycosidase, a kinase and an endonuclease. In some embodiments, the protein-of-interest is a polymerase. In some embodiments, the exonuclease activity of the polymerase is disabled.

Embodiments of the present disclosure also include a system for direct electrical measurement of protein activity. In accordance with these embodiments, the system includes any of the devices described herein, a means for introducing a chemical entity that is capable of interacting with the protein-of-interest, a means for applying a voltage bias between the first and second electrodes that is 100mV or less, and a means for monitoring fluctuations that occur as the chemical entity interacts with the protein-of-interest.

### BRIEF DESCRIPTION OF THE DRAWINGS

FIG. 1: Representative schematic diagram illustrating the criteria for selecting attachment points to an enzyme, according to one embodiment of the present disclosure.

FIGS. 2A-2B: Representative schematic diagram illustrating the structure of biocytin (FIG. 2A) and carbamate-linked biotin-lysine (FIG. 2B). Linkage of the biotin head group to the lysine sidechain is observed at the NE of lysine either through a peptide bond (biocytin) or carbamate moiety.

FIG. 3: Representative schematic diagram illustrating the binding pocket of a modified Pyrrolysol t-RNA synthetase bound to biocytin, according to one embodiment of the present disclosure.

FIG. 4: Representative schematic diagram illustrating expression of a polymerase containing biocytin, according to one embodiment of the present disclosure.

FIG. 5: Representative schematic diagram illustrating an electrical junction using a biocytin modified polymerase and trans divalent streptavidin, according to one embodiment of the present disclosure.

FIG. 6: Representative map of the cloned plasmid for the dual expression of Py1RS and Phi29. The gene encoding the Py1RS (orange) is controlled by the AraC promoter, while the Phi29 gene (blue) is controlled by lad promoter.

FIG. 7: Representative flow chart depicting the workflow for either single (left), or double incorporation (right) of the carbamate linked biotin-lysine in the production of dual biotinylated polymerase.

FIG. 8: Representative model of the dual biotinylated Phi29 polymerase. Incorporation of the carbamate linked biotin-lysine is depicted at the original lysine site for the N-terminal Avitag (blue) and position W274 (purple) in the mature, native Phi29 sequence.

FIGS. 9A-9C: Representative chemical reactions used to generate carbamate linked biocytin, according to one embodiment of the present disclosure.

FIGS. 10A-10C: Representative mass spectrometry data (MALDI) demonstrating the presence of each of the reaction products corresponding to FIGS. 9A-9C, respectively.

### DETAILED DESCRIPTION

Embodiments of the present disclosure include devices, systems, and methods related to protein bioelectronics. In particular, the present disclosure provides devices, systems, and methods for forming electrical contacts to a protein with high yield, which facilitates the manufacture of analytical devices to detect and measure the electrical characteristics corresponding to protein function.

In accordance with these embodiments, a peptide sequence capable of enzymatic recognition and modification is incorporated at two widely separated points on the enzyme, each chosen so as not to interfere with the function of the enzyme. In one embodiment, a polymerase (e.g., Φ29 polymerase) can be used as an enzyme into which, for example, an Avitag sequence can be inserted. (The Avitag sequence generally comprises the following amino acid sequence: GLNDIFEAQKIEWHE (SEQ ID NO: 1).) As is disclosed in more detail in PCT Application No. PCT/US2019/032707, which is incorporated herein by reference in its entirety and for all purposes, at the N terminus and at a point some 5 nm distant from the N terminus in the deactivated exonuclease domain of the polymerase. In some embodiments, the Avitag sequence can be biotinylated using the BirA enzyme. The resulting, doubly biotinylated polymerase can be self-assembled into an electronic junction using a pair of electrodes that have been coated with streptavidin, after the electrodes were first functionalized with thiolated biotin molecules.

A device configured as described above can be used for direct measurement of protein activity. In some embodiments, the device produces characteristic signals when the polymerase is activated in the presence of template DNA, primer DNA, and magnesium. However, the processivity of the polymerase and strand displacement activity can be improved. In some embodiments, and as provided further herein, the device can be improved, for example, but insertion of an Avitag sequence into various other locations within the enzyme (e.g., in locations other than the exonuclease domain). In one embodiment, improved activity was demonstrated by inserting a single modified amino acid, for example, an 4-Azido-L-phenylalanine as disclosed in more detail in PCT Application No. PCT/US2020/015931, which is incorporated herein by reference in its entirety and for all purposes. However, one limitation of this approach is that the conditions required for the subsequent click chemistry are somewhat harsh and can result in low yields on a biotinylated enzyme. Additionally, the use of streptavidin, which has four binding sites, results in a number of possible (and different) binding geometries. As described further herein, a simple method for directly incorporating biotin molecules at the desired attachment points in a protein-of-interest for establishing a well-defined connection between a biotinylated protein-of-interest and the electrodes would lead to improvements in performance and manufacturing.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As noted herein, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, compositions, systems and apparatuses/devices which may further include any and all elements from any other disclosed methods, compositions, systems, and devices, including any and all elements corresponding to detecting one or more target molecules (e.g., DNA, proteins, and/or components thereof). In other words, elements from one or another disclosed embodiments may be interchangeable with elements from other disclosed embodiments. Moreover, some further embodiments may be realized by combining one and/or another feature disclosed herein with methods, compositions, systems and devices, and one or more features thereof, disclosed in materials incorporated by reference. In addition, one or more features/elements of disclosed embodiments may be removed and still result in patentable subject matter (and thus, resulting in yet more embodiments of the subject disclosure). Furthermore, some embodiments correspond to methods, compositions, systems, and devices which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

Also, while some of the embodiments disclosed are directed to detection of a protein molecule, within the scope of some of the embodiments of the disclosure is the ability to detect other types of molecules.

When describing the molecular detecting methods, systems and devices, terms such as linked, bound, connect, attach, interact, and so forth should be understood as referring to linkages that result in the joining of the elements being referred to, whether such joining is permanent or potentially reversible. These terms should not be read as requiring a specific bond type except as expressly stated.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### 2. PROTEIN BIOELECTRONIC DEVICES

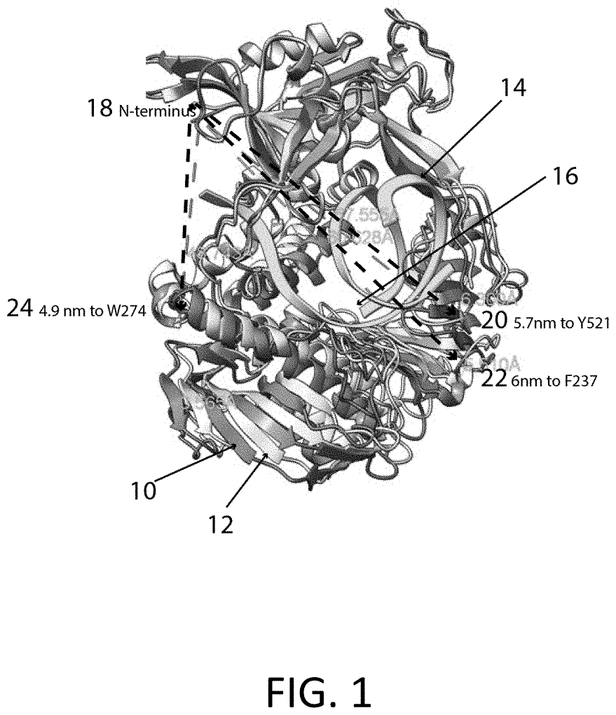

Embodiments of the present disclosure include methods of modifying a protein-of-interest (e.g., an enzyme) so as to allow for two points of electrical contact. Two exemplary structures of DNA polymerase Φ29 are shown superimposed in FIG. 1. The darker structure is pre-translocation, and the lighter structure is post translocation. The relative movement of the enzyme between these states is illustrated by the displacement of the two structures. This is illustrated by a region 10 that is displaced substantially 12 post translocation. The criteria for choosing connection points include, but are not limited to, the following: (1) that they are far from the active site of the enzyme; (2) that they are at points that do not move substantially as the enzyme undergoes functional motions; (3) that they are located on an accessible surface of the enzyme; and (4) that they widely separated, preferably by at least 5 nm if the overall size of the enzyme permits.

Referring to 14 in FIG. 1, the double-stranded region of the DNA template-primer complex is shown in this exemplary embodiment, with the junction between the double- and single-stranded regions 16 being the active site of the enzyme. The N-terminus of the enzyme 18 is in the exonuclease domain; it is not involved in the polymerase activity of the enzyme, and it is located at a position that is non-adjacent to the active site of the enzyme. Given this, this location was chosen as an first attachment point in this embodiment of the present disclosure.

Referring to 20, 22 and 24 in FIG. 1, the sites Y521, F237 and W274 are highlighted, respectively. Each of these sites is located at a position that is non-adjacent to the active site of the enzyme, and are at points that undergo minimal displacement (e.g., less than 0.5 nm) over the open to closed transition of the enzyme. Additionally, they are located on the surface of the enzyme and are approximately 5 nm or more from the N terminus (20 is 5.7 nm from the N terminus, 22 is 6 nm from the N terminus, and 24 is 4.9 nm from the N-terminus). Single amino-acid modifications at each of these sites do not interfere with enzyme activity and leave the processivity and strand displacement activity of the polymerase unaltered and functional. Accordingly, in some embodiments, these are all useful as second connection points, and electrical tests have shown that the conductivity of the enzyme attached to point 18 and any one of points 20, 22 and 24 is strongly modulated by enzyme activity, as the enzyme undergoes the open to closed conformational transition.

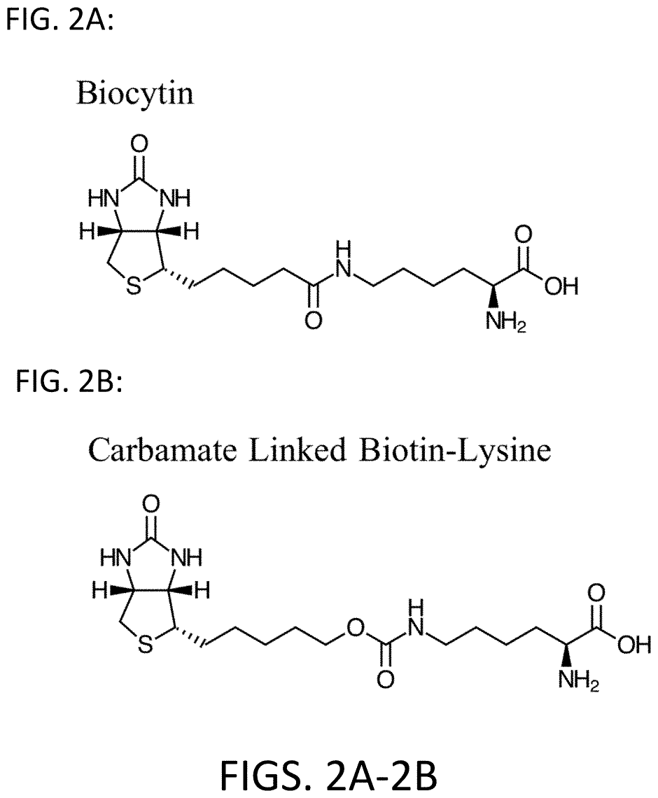

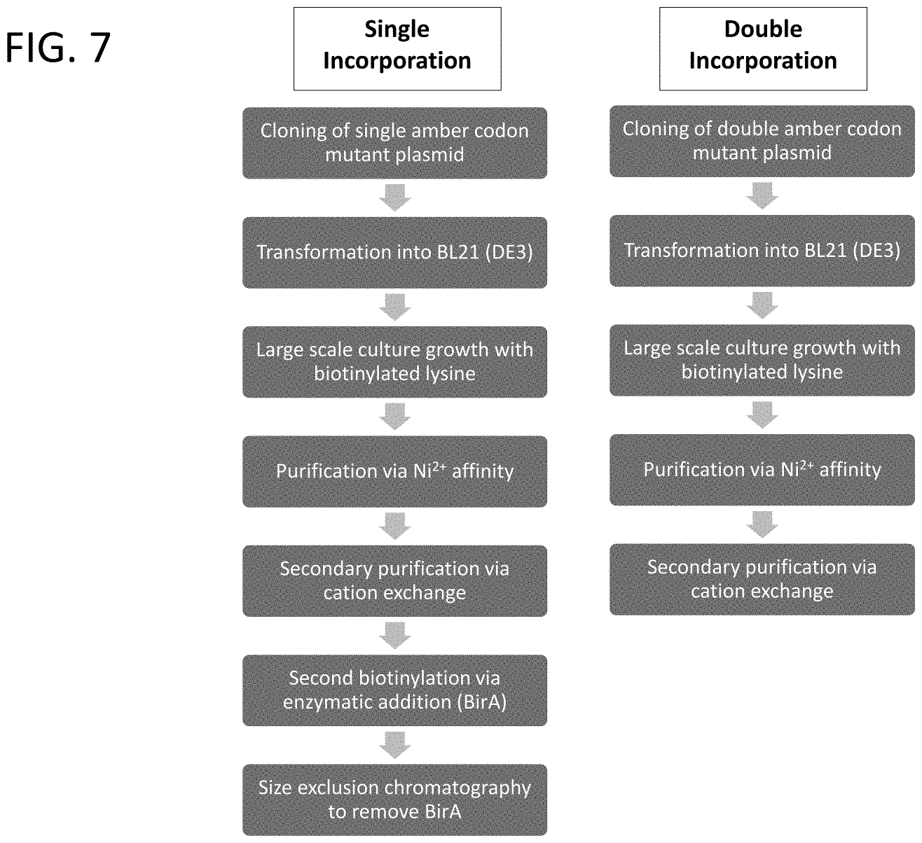

As described further herein, embodiments of the present disclosure include the use of one or more non-canonical amino acid substitutions in a protein-of-interest to enable a desired function (e.g., attachment point for an electrical connection). In some embodiments, the use of one or more non-canonical amino acids facilitates biotinylation of these sites in one step, as the enzyme is expressed (see, e.g., FIG. 7). For example, as shown in FIG. 2, the non-canonicalamino acid to be incorporated into a protein-of-interest is a biotinylated derivative of lysine, referred to as biocytin (biotinylated L-Lysine). Incorporation of this non-canonical amino acid results in a biotinylated lysine with the same structure as would result from the biotinylation of the lysine in the Avitag sequence by the BirA enzyme. Additionally, as shown in FIG. 2, this particular non-canonical amino acid differs from that of the natural metabolite Biocytin in that the biotin head group and lysine sidechain are linked via a carbamate functional group at the NE of lysine (FIG. 2B). Here, the carbamate moiety confers an additional degree of rotational restriction within the amino acid sidechain, as well as providing increased chemical and proteolytic stability. In addition, the carbamate group offers more intermolecular contact with the current pyrrolysyl tRNA synthetase through its increased hydrogen bonding potential. In some embodiments, the protein-of-interest can include biocytin and/or a biocytin derivative (e.g., carbamate linked biocytin). In some embodiments, the protein-of-interest can include biocytin and/or a biocytin derivative (e.g., carbamate linked biocytin) that has been incorporated through the use of an Avitag. In some embodiments, the protein-of-interest can include biocytin and/or a biocytin derivative (e.g., carbamate linked biocytin) that has been directly incorporated into the protein-of-interest during protein expression (e.g., does not involve the use of an Avitag polypeptide).

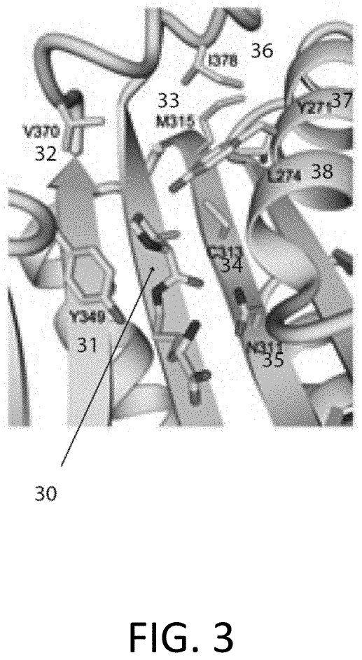

In some embodiments, insertion of a non-canonical amino acid(s) is achieved by repurposing a stop codon through the use of a modified t-RNA. For example, Hohl et al. (Hohl, A.; Karan, R.; Akal, A.; Renn, D.; Liu, X.; Ghorpade, S.; Groll, M.; Rueping, M.; Eppinger, J., Engineering a Polyspecific Pyrrolysyl-tRNA Synthetase by a High Throughput FACS Screen. Sci Rep 2019, 9 (1), 11971)) have described modifications to a polyspecific Pyrrolysol t-RNA synthetase that allows it to bind and incorporate a biocytin molecule. Referring to FIG. 3, the biocytin amino acid 30 is shown in the binding pocket of the modified Pyrrolysol t-RNA synthetase where the altered residues are indicated by 31-38.

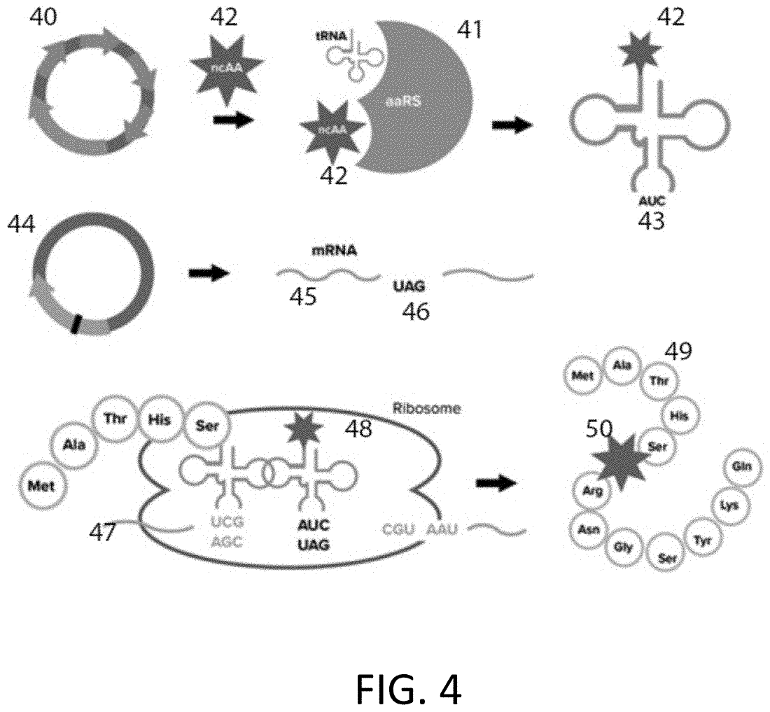

A procedure for expressing the modified Φ29 enzyme is illustrated in FIG. 4. A plasmid expression system 40 containing the cloned sequence for the modified Pyrrolysol t-RNA synthetase is used to express the synthetase 41 in the presence of biocytin 42. The product is a t-RNA 43 loaded with biocytin and containing the complement of a stop codon, AUC. In some embodiment, the same expression system also contains a plasmid with the sequence for the modified Φ29 enzyme with the complementary DNA sequence TAG at the sites where biocytin incorporation is desired (e.g., the N-terminus and W274, Y521 or F237 in the example discussed above). The messenger RNA 45 translated from this plasmid will contain the stop-codon sequence UAG 46 at sites where biocytin is to be incorporated. In the presence of an excess of the biocytin-bearing t-RNA 43, the ribosome 48 does not stop at the UAG codon, but rather inserts a biocytin amino acid. The result is a protein 49 incorporating the modified amino acid 50 at the desired locations. Since no chemical modification of the polymerase is required post-expression, and the incorporation of the biotin at the two desired sites is 100%, a greatly improved yield and greatly simplified production process are realized.

Embodiments of the present disclosure also includes a linker-protein used to tether the polymerase to the electrodes. Because of an abundance of surface cysteines, the polymerase Φ29 cannot contact the metal electrodes directly. Accordingly, linker proteins are used, as disclosed in more detail in PCT Application No. PCT/US2019/032707, which is incorporated herein by reference in its entirety and for all purposes. The strong and almost irreversible biotin streptavidin bond can be particularly advantageous. For example, electrodes are functionalized with a sulfur-terminated biotin molecule (as disclosed in the above reference) and then exposed to a solution of streptavidin molecules. The resulting streptavidin-coated electrodes are then exposed to a solution of the doubly-biotinylated polymerase, so that polymerase molecules can form bridges between the two electrodes by binding to the streptavidin molecules.

In some embodiments, the assembly of these junctions is a stochastic process, complicated by the 4-valent nature of streptavidin, as a variety of possible polymerase binding geometries are available, both cis (two binding sites on the same end of the molecule) and trans (at opposite ends of the molecule). Therefore, in some embodiments, a molecular wire with binding sites only at the N- and C-termini can be used, as disclosed in more detail in U.S. Provisional Patent Ser. No. 63/022,266, which is incorporated herein by reference in its entirety and for all purposes. This application discloses molecular wires of precisely controlled length and functionalization for wiring bioelectronic circuits.

However, in other embodiments, divalent streptavidin molecules can be generated that retain the highly cooperative binding of the 4-valent molecule. This can be achieved by assembling streptavidin from mixtures of dead (binding site disabled) and wild-type subunits, via chemical refolding, and separating fully-assembled streptavidin molecules of the desired stoichiometry using ion-exchange chromatography and charge-labeled tags on the subunits (see, e.g., Fairhead, M.; Krndija, D.; Lowe, E. D.; Howarth, M., Plug-and-play pairing via defined divalent streptavidins. J Mol Biol 2014, 426 (1), 199-214).

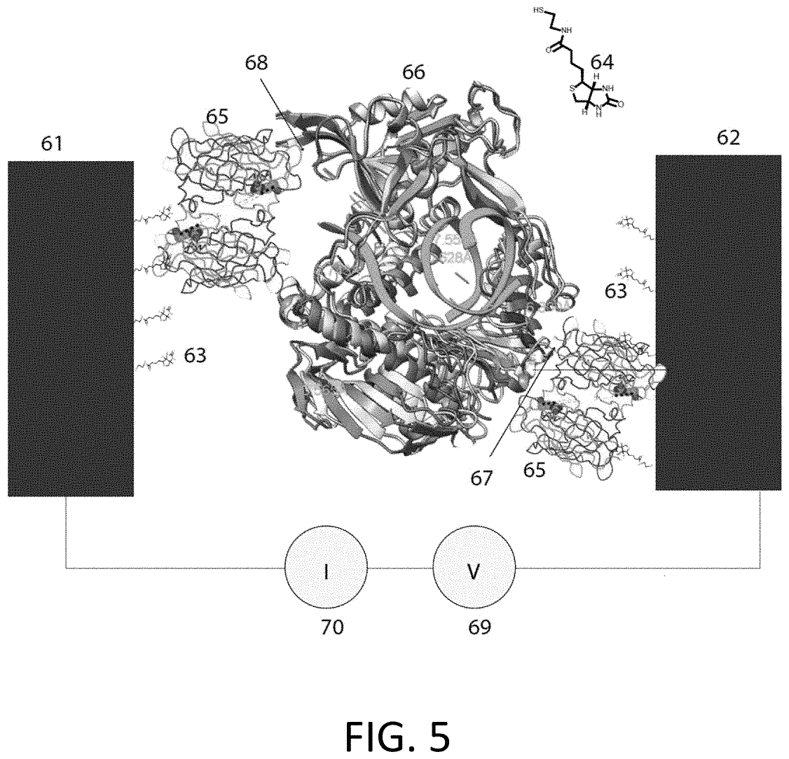

In accordance with these embodiments, the assembly of a junction proceeds as illustrated by the device shown in FIG. 5. A first electrode 61 and a second electrode 62 are functionalized with thiolated biotin molecules 63 (illustrated in a magnified structure as 64). The surfaces are then functionalized with trans divalent streptavidin 65. Introduction of the doubly biotinylated polymerase Φ29 66 results in structures that bridge the electrode gap via biotin binding to the trans sites indicated as 67 and 68. Applying a bias voltage (V) 69 results in a current flow (I) 70 through the polymerase, and fluctuations in this current will report on structural fluctuations of the polymerase.

As described further herein, embodiments of the present disclosure include a method of manufacturing a device for direct measurement of protein activity. In some embodiments, the method includes combining a first and second electrode with a protein-of-interest to form an electrical connection between the electrodes. The first and second electrodes comprise surfaces that are chemically modified with a linker molecule. In some embodiments, the surfaces of the first and second electrodes are chemically modified with at least one thiolated biotin linker molecule. In some embodiments, applying a voltage bias to the electrodes produces current flow through the protein-of-interest, and fluctuations in activity of the protein-of-interest correspond to fluctuations in current.

In some embodiments, the protein-of-interest comprises at least one non-canonical amino acid. Although the protein-of-interest can comprise any non-canonical amino acid (see, e.g., Quast, R. B., Cotranslational incorporation of non-standard amino acids using cell-free protein synthesis. FEBS Letters 2015, 589 (15), 1703-1712)), in some embodiments, the non-canonical amino acid comprises biotin or a derivative thereof. In some embodiments, the non-canonical amino acid is biocytin or a derivative thereof. In some embodiments, the protein-of-interest comprises two biocytin non-canonical amino acids. In some embodiments, the protein-of-interest comprises 2, 3, 4, 5, 6, 7, 8, 9, or 10 non-canonical amino acids.

In some embodiments, the protein-of-interest comprises an Avitag sequence (GLNDIPBAQKIEWHE (SEQ ID NO: 1), and the biocytin is incorporated into the protein-of-interest using the Avitag sequence. In some embodiments, the protein-of-interest does not comprise an Avitag sequence, and the biocytin is incorporated into the protein-of-interest directly during protein expression (see, e.g., FIG. 4) using tRNA synthetase. In some embodiments, the protein-of-interest includes at least one biocytin incorporated via the Avitag sequence, and at least one additional biocytin incorporated directly via tRNA synthetase.

In some embodiments, the protein-of-interest comprises the least one non-canonical amino acid at two distinct locations. In some embodiments, the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

In some embodiments, the protein-of-interest is selected from the group consisting of a polymerase, a nuclease, a proteasome, a glycopeptidase, a glycosidase, a kinase and an endonuclease. In some embodiments, the protein-of-interest is a polymerase. In some embodiments, the exonuclease activity of the polymerase is disabled.

In some embodiments, the method further comprises adding a second linker molecule to form the electrical connection. In some embodiments, the second linker molecule comprises a streptavidin molecule. In some embodiments, the streptavidin molecule comprises at least two biotin binding sites (see, e.g., FIG. 5).

Embodiments of the present disclosure also include a device for direct measurement of protein activity. In accordance with these embodiments, the device includes a first electrode and a second electrode, and the first and second electrodes comprise surfaces chemically modified with at least one thiolated biotin linker molecule. The device also includes a protein-of-interest that comprises at least one non-canonical amino acid, and the protein-of-interest is capable of forming an electrical connection between the first and second electrodes. In some embodiments, applying a voltage bias to the electrodes produces current flow through the protein-of-interest, and fluctuations in activity of the protein-of-interest correspond to fluctuations in current.

In some embodiments of the device, the non-canonical amino acid comprises biotin or a derivative thereof. In some embodiments, the non-canonical amino acid is biocytin or a derivative thereof. In some embodiments, the protein-of-interest comprises two biocytin non-canonical amino acids. In some embodiments, the protein-of-interest comprises 2, 3, 4, 5, 6, 7, 8, 9, or 10 non-canonical amino acids. As would be understood by one of ordinary skill in the art based on the present disclosure, the protein-of-interest can comprise any non-canonical amino acid (see, e.g., Quast, R. B., Cotranslational incorporation of non-standard amino acids using cell-free protein synthesis. FEBS Letters 2015, 589 (15), 1703-1712)), including but not limited to, biocytin and biocytin derivatives.

In some embodiments of the device, the protein-of-interest comprises an Avitag sequence (GLNDIFEAQKIEWHE (SEQ ID NO: 1), and the biocytin is incorporated into the protein-of-interest using the Avitag sequence. In some embodiments, the protein-of-interest does not comprise an Avitag sequence, and the biocytin is incorporated into the protein-of-interest directly during protein expression (see, e.g., FIG. 4) using tRNA synthetase. In some embodiments, the protein-of-interest includes at least one biocytin incorporated via the Avitag sequence, and at least one additional biocytin incorporated directly via tRNA synthetase. In some embodiments of the device, the protein-of-interest comprises the least one non-canonical amino acid at two distinct locations. In some embodiments, the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

In some embodiments, the device further comprises a second linker molecule comprising a streptavidin molecule. In some embodiments, the second linker molecule comprises a streptavidin molecule. In some embodiments, the streptavidin molecule comprises at least two biotin binding sites (see, e.g., FIG. 5).

In some embodiments of the device, the protein-of-interest is selected from the group consisting of a polymerase, a nuclease, a proteasome, a glycopeptidase, a glycosidase, a kinase and an endonuclease. In some embodiments, the protein-of-interest is a polymerase. In some embodiments, the exonuclease activity of the polymerase is disabled.

### 3. SYSTEMS AND METHODS

Embodiments of the present disclosure also include a system for direct electrical measurement of protein activity. In accordance with these embodiments, the system includes any of the devices described herein, a means for introducing a chemical entity that is capable of interacting with the protein-of-interest, a means for applying a voltage bias between the first and second electrodes that is 100 mV or less, and a means for monitoring fluctuations that occur as the chemical entity interacts with the protein-of-interest.

Embodiments of the present disclosure also include an array comprising a plurality of any of the bioelectronic devices described herein. In some embodiments, the array includes a means for introducing an analyte capable of interacting with the protein, a means for applying a voltage bias between the first and second electrodes that is 100mV or less, and a means for monitoring fluctuations that occur as the chemical entity interacts with the protein. The array can be configured in a variety of ways, as would be appreciated by one of ordinary skill in the art based on the present disclosure.

Embodiments of the present disclosure also include methods of measuring electronic conductance through a protein using any of the devices and systems described herein. In accordance with these embodiments, the present disclosure includes methods for direct electrical measurement of protein activity. In some embodiments, the method includes introducing an analyte capable of interacting with the protein to any of the bioelectronic devices described herein, applying a voltage bias between the first and second electrodes that is 100mV or less, and observing fluctuations in current between the first and second electrodes that occur when the analyte interacts with the protein. In some embodiments, the analyte is a biopolymer selected from the group consisting of a DNA molecule, an RNA molecule, a peptide, a polypeptide, and a glycan. In some embodiments, methods of the present disclosure include use of the devices and systems described herein to sequence a biopolymer. In some embodiments, the present disclosure includes methods for sequencing a polynucleotide using a bioelectronic device that obtains a bioelectronic signature of polymerase activity based on current fluctuations as complementary nucleotidepolyphosphate monomers are incorporated into the template polynucleotide.

As described further herein, the devices, systems, and methods of the present disclosure can be used to generate a bioelectronic signature of an enzyme-of-interest, which can be used to determine the sequence of any biopolymer (e.g., polynucleotide). In some embodiments, the enzyme-of-interest can be a polymerase, and various aspects of a bioelectronic signature of a polymerase as it adds nucleotide monomers to a template polynucleotide strand can be used to determine the sequence of that template polynucleotide. For example, a bioelectronic signature of polymerase activity can be based on current fluctuations as each complementary nucleotide monomer is incorporated into the template polynucleotide. In some embodiments, the bioelectronic device used to generate a bioelectronic signature comprises a polymerase functionally coupled to both a first electrode and a second electrode using the adaptor polypeptides of the present disclosure. The term “nucleotide” generally refers to a base-sugar-phosphate combination and includes ribonucleoside triphosphates ATP, UTP, CTG, GTP and deoxyribonucleoside triphosphates such as dATP, dCTP, dITP, dUTP, dGTP, dTTP, or derivatives thereof.

As one of ordinary skill in the art will readily recognize and appreciate after having benefited from the teachings of the present disclosure, the methods described herein can be used with any bioelectronic device that senses the duration of the open and closed states of an enzyme (e.g., polymerase). Exemplary devices include, but are not limited to, the bioelectronic devices and systems disclosed in U.S. Pat. No. 10,422,787 and PCT Appin. No. PCT/US2019/032707, both of which are herein incorporated by reference in their entirety and for all purposes. Additionally, it will be readily recognized and appreciated by those of ordinary skill in the art based on the present disclosure that the forgoing embodiments apply equally to (and include) sequencing RNAs with the substitution of rNTPs for dNTPs and the use of an RNA polymerase.

Further, one of ordinary skill in the art would readily recognize and appreciate that the methods described herein can be used in conjunction with other methods involving the sequencing of a biopolymer. In particular, the various embodiments disclosed in PCT Application No. PCT/US21/19428, which is herein incorporated by reference in its entirety, describes the interpretation of current fluctuations generated by a DNA polymerase as it actively extends a template, and how signal features (e.g., bioelectronic signature) may be interpreted in terms of the nucleotide being incorporated, and thus, how these signals can read the sequence of the template. This approach utilizes features of the signal that vary in time. For example, the time that the polymerase stays in a low current state reflects the concentration of the nucleotidetriphosphate in solution. If the concentration of a particular nucleotide triphosphate is low, then the polymerase must stay open for a longer time in order to capture the correct nucleotide, and since the open conformation of the polymerase corresponds to a lower current, the dip in current associated with the open state lasts for longer. Additionally, the various embodiments disclosed in PCT Application No. PCT/US20/38740, which is herein incorporated by reference in its entirety, describes how the base-stacking polymerization rate constant differences are reflected in the closed-state (high current states) so that the duration of these states may also be used as an indication of which one of the four nucleotides is being incorporated. It can be desirable to be able to use the amplitude of the signal as yet an additional contribution to determining sequence. Further, the various embodiments disclosed in PCT Application No. PCT/US21/17583, which is herein incorporated by reference in its entirety, describes methods that utilize a defined electrical potential to maximize electrical conductance of a protein-of-interest (e.g., polymerase), which can serve as a basis for the fabrication of enhanced bioelectronic devices for the direct measurement of protein activity. Additionally, the various embodiments disclosed in PCT Application No. PCT/US21/30239, which is herein incorporated by reference in its entirety, describes methods for sequencing a polynucleotide using a bioelectronic device that obtains a bioelectronic signature of polymerase activity based on current fluctuations as complementary nucleotidepolyphosphate monomers having distinctive charges are incorporated into the template polynucleotide.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

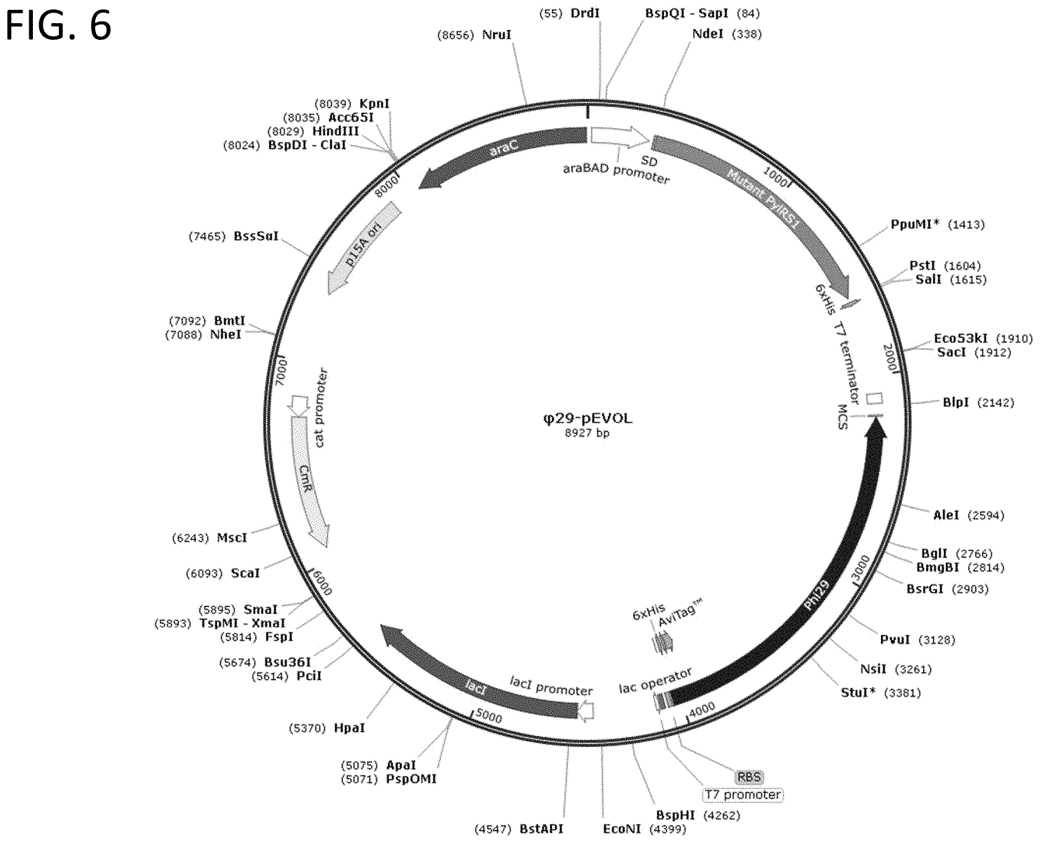

Experiments were conducted to generate the bioelectronic devices of the present disclosure using direct incorporation of the biotinylated lysine. Direct incorporation proceeds using a co-evolved pyrrolysyl tRNA synthetase (Py1RS)/tRNA pair from the bacterium M. Barkeri. For the site-specific incorporation into the target gene, and dual expression of the Py1RS/tRNA pair, a single plasmid containing the expression genes for Phi29 (controlled by the lad operon) and Py1RS (controlled by the AraC operon) was created (FIG. 6). Using the aforementioned plasmid, dual biotinylation of the polymerase can be achieved following either a single or double insertion of the biotin-lysine from two distinct protocols (FIG. 7). 100771 For a single incorporation of the non-canonical biotin-lysine amino acid, a single amber codon is inserted at one of the defined mutation sites (e.g., Y521, W274, or F237) from the mature Phi29 protein sequence. Fully functional polymerase with the incorporated biotin-lysine amino acid is expressed in liquid culture medium directly supplemented with the biotin-lysine derivative (˜400 mg/L). Purification of the incorporated product is carried out via Ni²+affinity chromatography, followed by cation exchange chromatography. The purified product is then subjected to BirA enzyme treatment to add a second biotin on the N-terminus via AviTag. Removal of residual BirA enzyme from the final dual-biotin polymerase is achieved through size-exclusion chromatography.

### Example 2

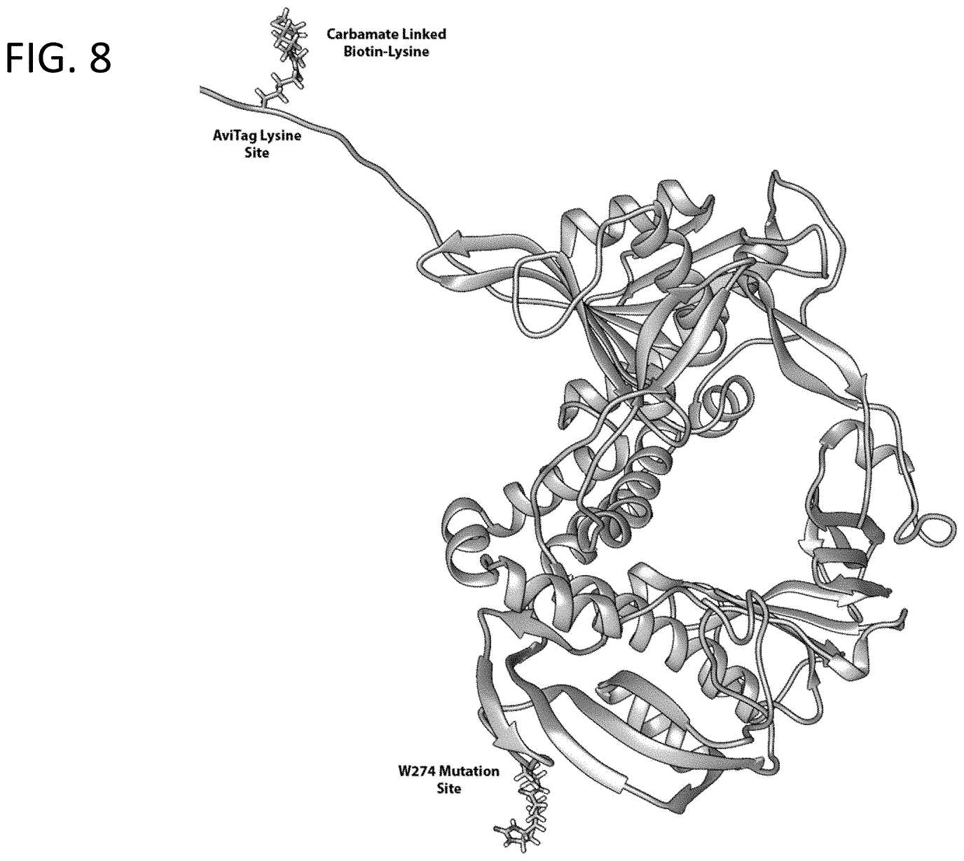

Experiments were conducted to generate the bioelectronic devices of the present disclosure using double insertion of the biotin-lysine amino acid. This procedure follows much of the same procedure for the single incorporation, in which the polymerase is expressed in liquid medium containing the amino acid derivative and purified via Ni2+ and cation exchange chromatography. To preserve as much similarity as possible to the biotinylation sites in the single incorporation protocol, a gene construct of the Phi29 polymerase will be made to include an additional amber codon at the exact site of the BirA targeted lysine in the N-terminal AviTag sequence. Here, rather than enzymatic addition, incorporation of biotin will be achieved at the exact same site as previously described but now through direct incorporation during protein expression. In this case, dual-biotin polymerase is produced through a simple “one-step” expression system and does not require additional enzymatic treatment, nor further separation through additional chromatography. A representative flow-chart of the two incorporation protocols can be viewed in FIG. 7. In addition, a model of the double incorporation of Phi29 polymerase can be seen in FIG. 8.

### Example 3

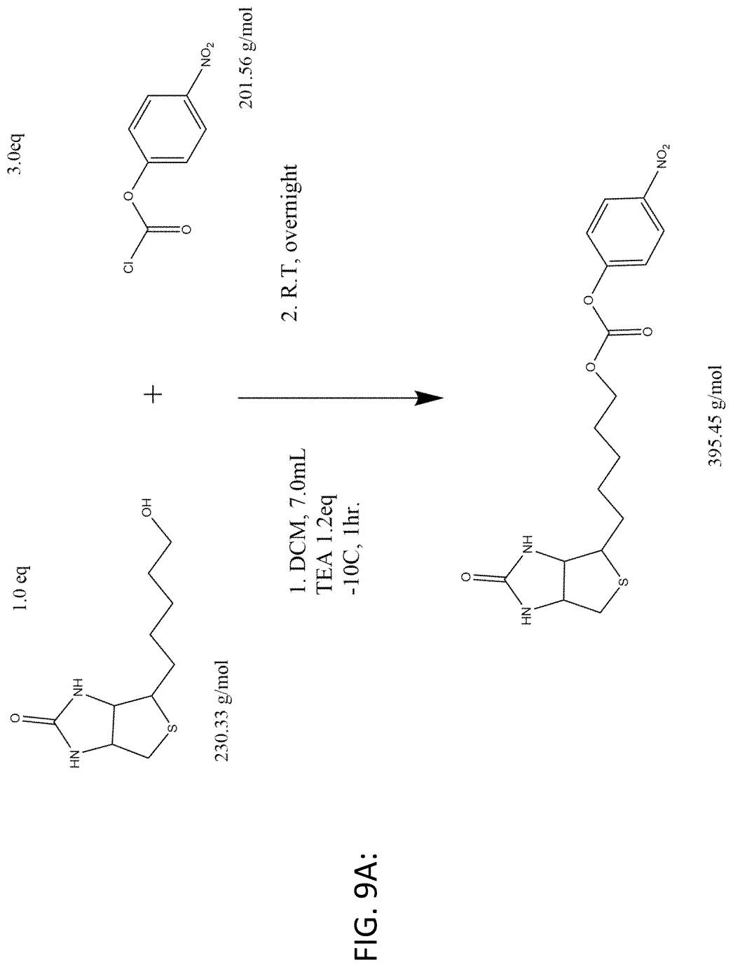

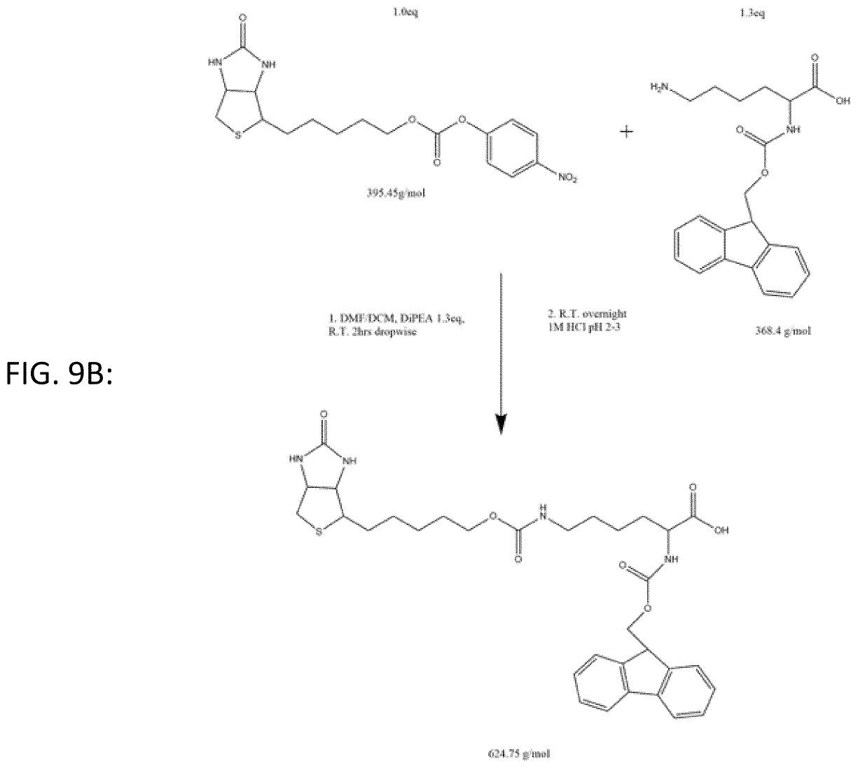

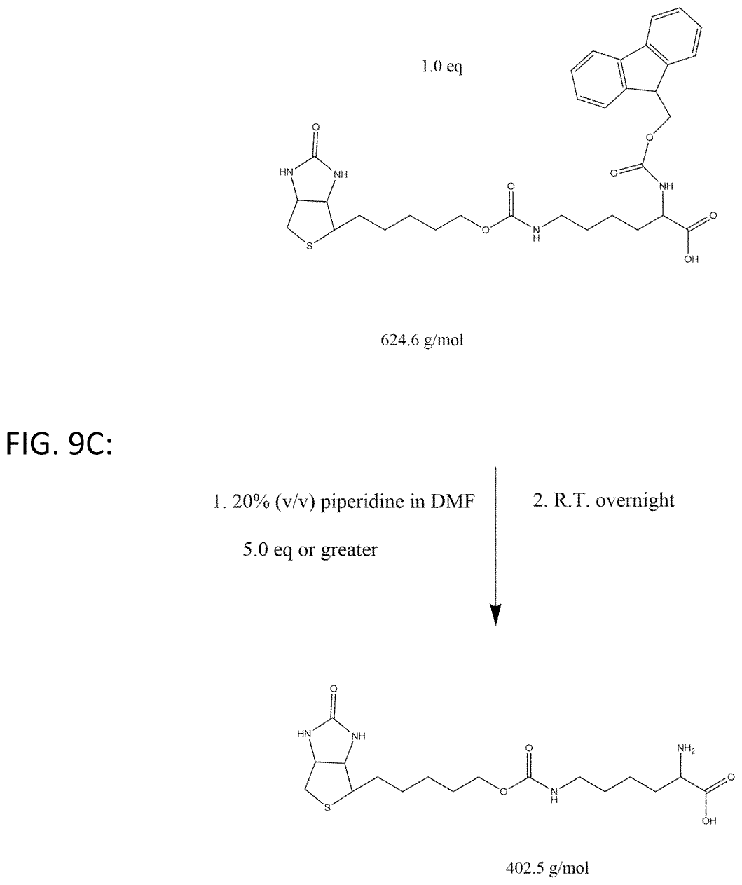

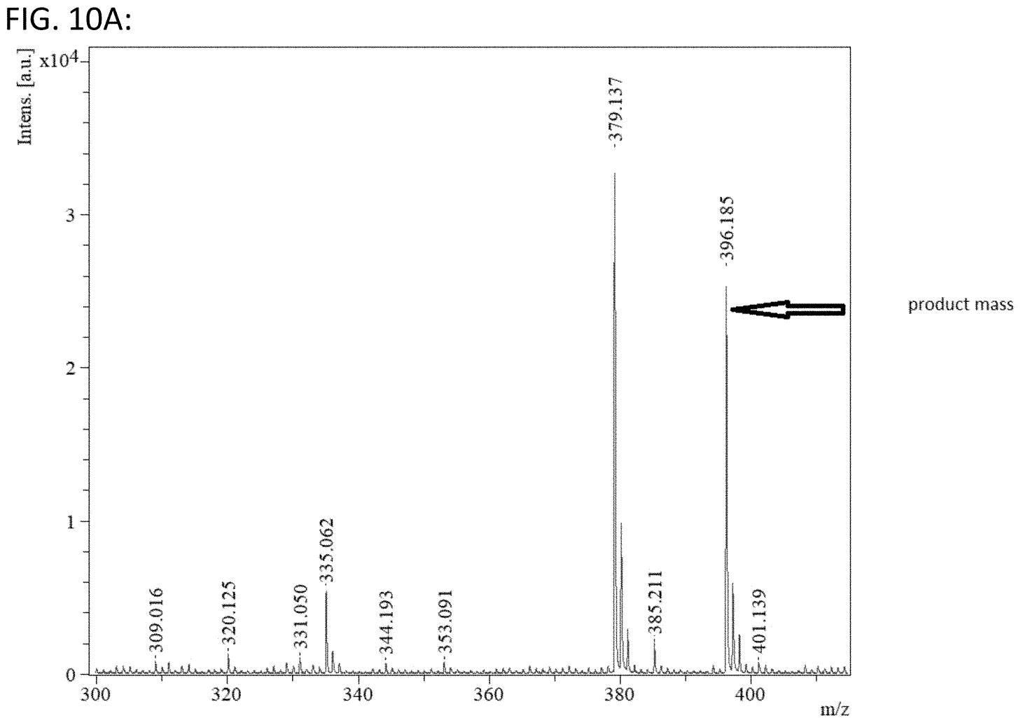

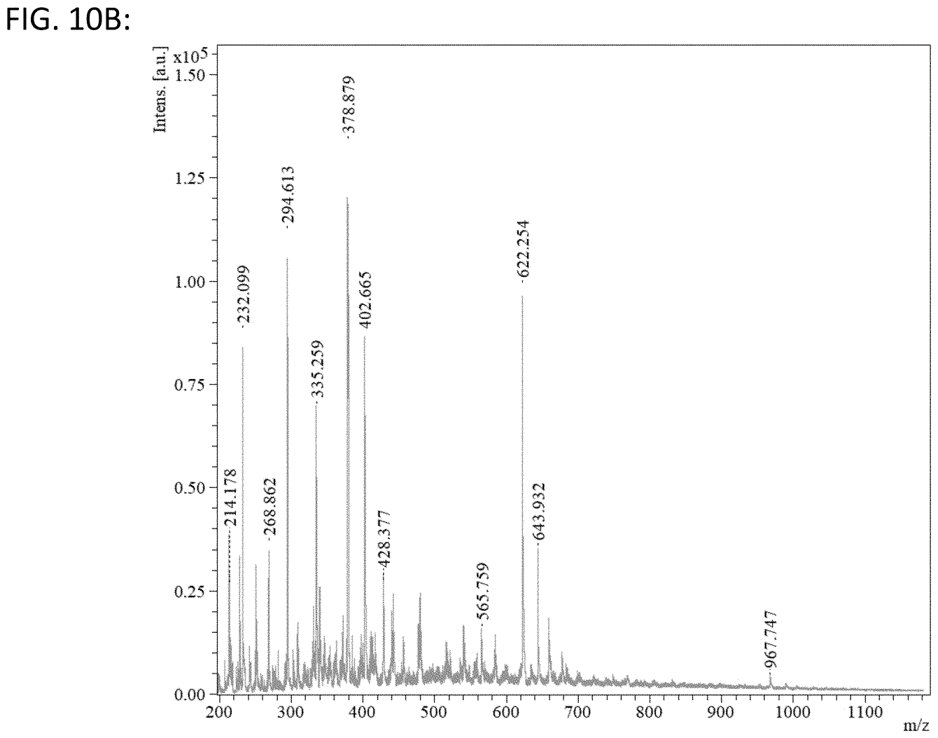

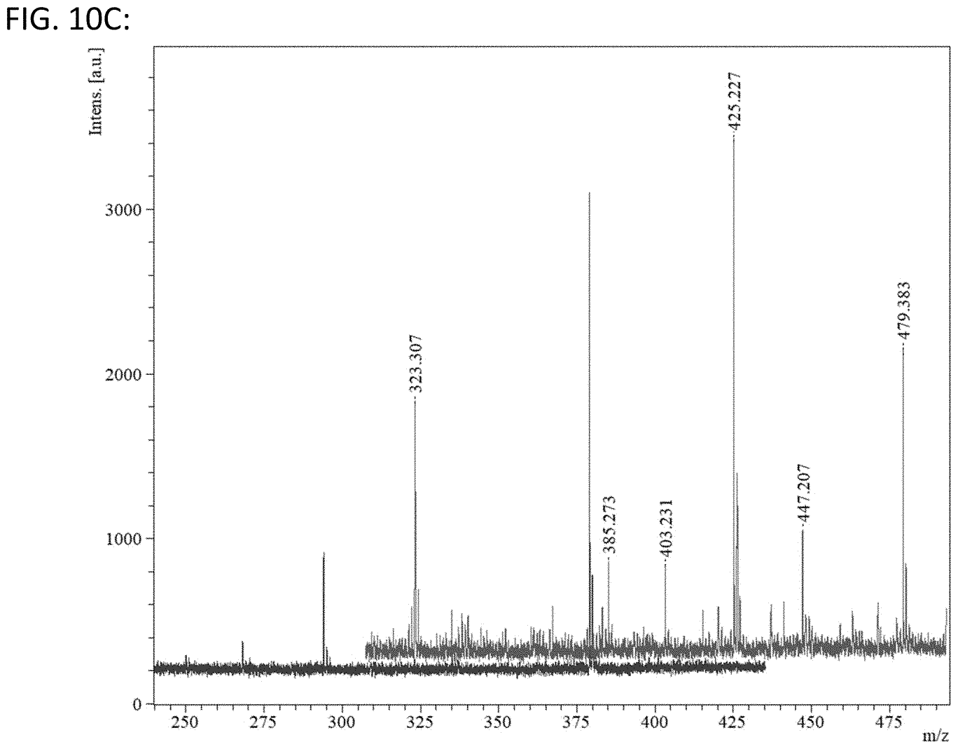

Experiments were conducted to generate the bioelectronic devices of the present disclosure using methods involving the direct incorporation of a non-canonical amino acid. As shown in FIGS. 9A-9C, various reactions were conducted to synthesize the carbamate linked biocytin, which can be directly incorporated into a protein-of-interest using existing tRNA synthetase enzymes. The first reaction is shown in FIG. 9A, the second reaction is shown in FIG. 9B, and the third reaction is shown in FIG. 9C. Corresponding mass spectrometry data (MALDI) demonstrating the presence of each of the reaction products are shown in FIGS. 10A-10C, respectively.

With respect to the first reaction (FIG. 9A), DCM and DMF were dried overnight over regenerated molecular sieves which had been heated in a drying oven at 175° C. for at least 4 hrs. Once dry, 7.0 mL of DCM was added to a 25 mL Schlenk flask w/stir bar and chilled to −10° C. with an ice and salt bath, and held there for a minimum of 20 mins. 4-nitrophenyl chloroformate (1.05 g, 5.22 mmol) was slowly added in portions to the chilled DCM under nitrogen flow before being capped with rubber septum. After the chloroformate was added, a separate solution was made with (0.4 g, 1.73 mmol) biotinyl alcohol dissolved in 7.0 mL of a 50/50 (v/v) mixture of DMF and DCM. To this suspension, triethylamine (0.294 mL, 0.213 g, 2.1 mmol) was added before transferring the mixture into a pressure equalized addition funnel. The solution was then added dropwise to the chilled chloroformate suspension over the course of lhr, making sure the temperature did not rise above −10° C. for the entire addition. The flask was then removed from the ice/salt bath and allowed to warm to room temperature and stir overnight. The TLC was run in 5% methanol in DCM. The product was separated on a manual silica gel column equilibrating first with hexanes, then 100% DCM, then slowly the gradient was increased to 5% MeOH in DCM. The product came off between 2-4% MeOH in DCM concentration. The yield was 0.32 g, or 46.7%.

Representative mass spectrometry data (MALDI) demonstrating the presence of the reaction products is shown in FIG. 10A. The materials used are provided below in Table 1.

TABLE 1
Materials for reaction #1.

| | | Batch/Lot | | | Grams | Moles | | M.P. | B.P. |
|---|---|---|---|---|---|---|---|---|---|
| Name | Vendor | number | g/mole | g/cm³ | used | used | | ° C. | ° C. |
| N,N-Dimethyl | Sigma-Aldrich | SHBN069 | 73.1 | 0.948 | 3.32 | 45.30 | mmol | −60.5 | 153 |
| formamide | | | | | | | | | |
| Dichloromethane | VWR | 0000239887 | 84.93 | 1.32 | 13.86 | 163.19 | mmol | −96.7 | 39.6 |
| Biotinyl alcohol | 1Pluschem | M13946 | 230.3 | | 0.4 | 1.73 | mmol | N/A | N/A |
| 4-nitrophenyl | Sigma-Aldrich | HMBH8102 | 201.56 | | 1.05 | 5.22 | mmol | 78 | 160 |
| chloroformate | | | | | | | | | |
| triethylamine | SigmaAldrich | MKCP0112 | 101.19 | 0.7255 | 0.213 | 2.1 | mmol | −114 | 88.6 |

### Example 4

With respect to the second reaction (FIG. 9B), Fmoc-lys-OH (0.3 g, 0.814 mmol) was suspended in 4 mL of DCM that was dried over molecular sieves in a 25 mL schlenk flask under nitrogen. DiPEA (0.15 mL, 0.111 g, 0.85 mmol) was added to this suspension before capping with a rubber septum and setting aside. In a pressure equalized addition funnel, the previously obtained 4-nitrophenyl-biotinyl carbonate (0.25 g, 0.632 mmol)was dissolved in 4 mL of DMF dried over molecular sieves. This solution was the added dropwise to the Fmoc-lys solution at R.T. under nitrogen over the course of an hour. The reaction mix had all volatiles removed before separating on a silica column The column was equilibrated with hexanes, then with 100% DCM, before slowly increasing the gradient to 10% MeOH, increasing the gradient by 2% every 100 mL. The product eluted around 7-8% MeOH concentration. The TLC was run in 10% MeOH. The product had an Rf around 0.52 and was UV active on the TLC plate.

Representative mass spectrometry data (MALDI) demonstrating the presence of the reaction products is shown in FIG. 10B. The target mass is about 624.6 g/mol. The peak at 622.2 is indicative of the product minus the 2 labile amine hydrogens on the lysine sidechain and peptide backbone. The peak at 644.0 is close to the mass for the sodium adduct of this product. The materials used are provided below in Table 2.

TABLE 2
Materials for reaction #2.

| | | Batch/Lot | | | Grams | Moles | M.P. | B.P. |
|---|---|---|---|---|---|---|---|---|
| Name | Vendor | number | g/mole | g/cm³ | used | used | ° C. | ° C. |
| N,N-Dimethyl | Sigma-Aldrich | SHBN069 | 73.1 | 0.948 | | | −60.5 | 153 |
| formamide | | | | | | | | |
| Dichloromethane | VWR | 0000239887 | 84.93 | 1.32 | | | −96.7 | 39.6 |
| F-moc-lys-OH | AmBeed | A186089-012 | 368.43 | 1.2 | | | 175 | 607 |
| DiPEA | Sigma-Aldrich | SHBM7942 | 129.24 | 0.742 | | | −50 | 127 |

### Example 5

With respect to the second reaction (FIG. 9C), about 0.51 g of N-biotinyl-Fmoc-Lysine was dissolved in 5 mL of 20% piperidine in DMF solution. This mixture was stirred at R.T. for 16 hrs under nitrogen in a 10 mL Schlenk flask. The reaction mix was then rotary evaporated until all solvent was removed. The residue was adhered to silica and separated on a silica column. The column was equilibrated with 100 mL of hexane, followed by 50 mL of 100% DCM. The gradient was then slowly increased by 4% MeOH every 50 mL. The product came off around 30% MeOH concentration. The final yield was 128mg, which corresponds to a yield of 39%. The TLC was run in 25% methanol in DCM. The product ran lower than the piperidine and its salts. With an Rf of about 0.29.

Representative mass spectrometry data (MALDI) demonstrating the presence of the reaction products is shown in FIG. 10C. The dark blue trace is the CHCA matrix which did have some slight overlap around 403 g/mol previously. The cyan trace is the product that was isolated. Results clearly demonstrate that the peaks at 403, 425, and 447 correspond to the sample and not the matrix. The peaks at 403, 425, and 447 correspond to the zero, single, and double sodium adducts of the product, respectively. The materials used are provided below in Table 3.

TABLE 3
Materials for reaction #3.

| | | Batch/Lot | | | Grams | Moles | M.P. | B.P. |
|---|---|---|---|---|---|---|---|---|
| Name | Vendor | number | g/mole | g/cm³ | used | used | ° C. | ° C. |
| N,N-Dimethyl | Sigma-Aldrich | SHBN069 | 73.1 | 0.948 | 3.79 | 51.8 mmol | −60.5 | 153 |
| formamide | | | | | | | | |
| Piperidine | Sigma-Aldrich | SHBK7500 | 85.15 | 0.862 | 0.862 | 10.1 mmol | −13.0 | 106 |

What is claimed is:

1. A method of manufacturing a device for direct measurement of protein activity, the method comprising:
combining a first and second electrode with a protein-of-interest to form an electrical connection between the electrodes, wherein the first and second electrodes comprise surfaces chemically modified with a linker molecule, and wherein the protein-of-interest comprises at least one non-canonical amino acid;
wherein applying a voltage bias to the electrodes produces current flow through the protein-of-interest.

2. The method of claim 1, wherein fluctuations in activity of the protein-of-interest correspond to fluctuations in current.

3. The method of claim 1, wherein the surfaces of the first and second electrodes are chemically modified with at least one thiolated biotin linker molecule.

4. The method of claim 1, wherein the at least one non-canonical amino acid comprises biotin or a derivative thereof.

5. The method of claim 4, wherein the at least one non-canonical amino acid is biocytin or a derivative thereof.

6. The method of claim 4, wherein the protein-of-interest comprises two biocytin non-canonical amino acids or derivatives thereof.

7. The method of claim 1, wherein the protein-of-interest comprises an Avitag sequence or a derivative thereof.

8. The method of claim 1, wherein the protein-of-interest does not comprise an Avitag sequence or a derivative thereof.

9. The method of claim 1, wherein the method further comprises adding a second linker molecule to form the electrical connection.

10. The method of claim 9, wherein the second linker molecule comprises a streptavidin molecule.

11. The method of claim 10, wherein the streptavidin molecule comprises at least two biotin binding sites.

12. The method of claim 1, wherein the protein-of-interest comprises the least one non-canonical amino acid at two distinct locations.

13. The method of claim 12, wherein the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

14. The method of claim 1, wherein the protein-of-interest is selected from the group consisting of a polymerase, a nuclease, a proteasome, a glycopeptidase, a glycosidase, a kinase and an endonuclease.

15. The method of claim 1, wherein the protein-of-interest is a polymerase.

16. The method of claim 15, wherein exonuclease activity of the polymerase is disabled.

17. A device for direct measurement of protein activity, the device comprising:
a first electrode and a second electrode, wherein the first and second electrodes comprise surfaces chemically modified with at least one thiolated biotin linker molecule; and
a protein-of-interest that forms an electrical connection between the first and second electrodes comprising at least one non-canonical amino acid, wherein the at least one non-canonical amino acid comprises biotin or a derivative thereof;
wherein applying a voltage bias to the electrodes produces current flow through the protein-of-interest.

18. The device of claim 17, wherein fluctuations in activity of the protein-of-interest correspond to fluctuations in current.

19. The device of claim 17, wherein the at least one non-canonical amino acid is biocytin or a derivative thereof.

20. The device of claim 17, wherein the protein-of-interest comprises two biocytin non-canonical amino acids or derivatives thereof.

21. The device of claim 17, wherein the protein-of-interest comprises an Avitag sequence or a derivative thereof.

22. The device of claim 17, wherein the protein-of-interest does not comprise an Avitag sequence or a derivative thereof.

23. The device of claim 17, wherein the device further comprises a second linker molecule comprising a streptavidin molecule.

24. The device of claim 17, wherein the protein-of-interest comprises the least one non-canonical amino acid at two distinct locations.

25. The device of claim 24, wherein the distinct locations comprise at least one of: (i) non-adjacent locations; (ii) locations that do not undergo substantial movement during protein activity; (iii) locations that are on an accessible surface of the protein-of-interest; and/or (iv) locations that are separated by at least 5 nm.

26. The device of claim 17, wherein the protein-of-interest is selected from the group consisting of a polymerase, a nuclease, a proteasome, a glycopeptidase, a glycosidase, a kinase and an endonuclease.

27. The device of claim 17, wherein the protein-of-interest is a polymerase.

28. The device of claim 27, wherein exonuclease activity of the polymerase is disabled.

29. A system for direct electrical measurement of protein activity, the system comprising:
the device of claim 17;
a means for introducing a chemical entity that is capable of interacting with the protein-of-interest;
a means for applying a voltage bias between the first and second electrodes that is 100 mV or less; and
a means for monitoring fluctuations that occur as the chemical entity interacts with the protein-of-interest.
